(19) United States
(12) Patent Application Publication
(10) Pub. No.: US 2018/0266983 A1
(43) Pub. Date: Sep. 20, 2018

(54) ELECTROCHEMICAL SENSOR

(71) Applicant: Honeywell International Inc., Morris Plains, NJ (US)

(72) Inventors: Yuzhong Yu, Shanghai (CN); Huafang Zhou, San Jose, CA (US); Na Wei, Shanghai (CN)

(21) Appl. No.: 15/754,254

(22) PCT Filed: Aug. 24, 2015
(86) PCT No.: PCT/US2015/046554

§ 371 Date: Feb. 21, 2018

(51) Int. Cl. G01N 27/404; G01N 33/00

(52) U.S. Cl. CPC G01N 27/4045; G01N 33/0044

(57) ABSTRACT

An electrochemical H₂S sensor comprises a housing, an electrolyte disposed within the housing, and a plurality of electrodes in contact with the electrolyte within the housing. The plurality of electrodes comprise a porous working electrode that comprises a first surface that is hydrophobic and a second surface that is treated with a surfactant. The first surface is exposed to an ambient gas, and the second surface treated with the surfactant is in contact with the electrolyte.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

In monitoring for the presence of hydrogen sulfide (H₂S), other gases such as carbon monoxide (CO) can be present. The additional gases can react at a working electrode in a hydrogen sulfide sensor. For example, the working electrode can comprise a noble metal that can catalyze the reaction of both hydrogen sulfide and carbon monoxide. As a result, the presence of carbon monoxide may create a cross-sensitivity in the hydrogen sulfide sensor, resulting in the false impression that greater levels of hydrogen sulfide are present in the ambient gases than are actually present. Due to the danger presented by the presence of hydrogen sulfide, the threshold level for triggering an alarm can be relatively low, and the cross-sensitivity due to the presence of the carbon monoxide may be high enough to create a false alarm for the hydrogen sulfide sensor.

Some sensors are corrected for cross-sensitivity by calibrating the sensor in the presence of multiple gases including CO and H₂S. The readings for the H₂S can be corrected to take into account the presence of the CO, which may result in an artificially low H₂S reading in the absence of CO. This low reading may create a safety hazard when H₂S is present in the gas being monitored.

### SUMMARY

In an embodiment, an electrochemical H₂S sensor comprises a housing, an electrolyte disposed within the housing, and a plurality of electrodes in contact with the electrolyte within the housing. The plurality of electrodes comprise a porous working electrode that comprises a first surface that is hydrophobic and a second surface that is treated with a surfactant. The first surface is exposed to an ambient gas, and the second surface treated with the surfactant is in contact with the electrolyte.

In an embodiment, an electrochemical H₂S sensor comprises a housing, an electrolyte disposed within the housing, a reference electrode disposed within the housing and in contact with the electrolyte, a counter electrode disposed within the housing and in contact with the electrolyte, and a porous working electrode. The porous working electrode comprises a substrate comprising carbon and a hydrophobic material. A first surface of the substrate is hydrophobic, and a second surface of the substrate opposite the first surface is treated with a fluorosurfactant. The first surface is exposed to an ambient gas, and the second surface treated with the fluorosurfactant is in contact with the electrolyte.

In an embodiment, a method of detecting hydrogen sulfide comprises receiving an ambient gas comprising hydrogen sulfide into a housing, contacting the ambient gas with a porous working electrode, allowing the hydrogen sulfide to diffuse through the working electrode to contact an electrolyte, generating a current between the working electrode and a counter electrode in response to a reaction between the hydrogen sulfide and the electrolyte at the second surface of the working electrode, and determining a concentration of the hydrogen sulfide in the ambient gas based on the current. The porous working electrode comprises a substrate comprising carbon and a hydrophobic material. A first surface of the substrate is hydrophobic, and a second surface of the substrate opposite the first surface is treated with a fluorosurfactant. The method can also include adsorbing the carbon monoxide on the carbon in the substrate, and preventing at least a portion of the carbon monoxide from contacting the electrolyte through the porous working electrode.

These and other features will be more clearly understood from the following detailed description taken in conjunction with the accompanying drawings and claims.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIG. 1. schematically illustrates a cross section drawing of an electrochemical sensor according to an embodiment.

FIG. 2. illustrates a sensor response to exposure to 25 ppm H₂S under the conditions described in Example 1.

FIG. 3. illustrates a sensor response to exposure to 50 ppm CO under the conditions described in Example 1.

### DETAILED DESCRIPTION

It should be understood at the outset that although illustrative implementations of one or more embodiments are illustrated below, the disclosed systems and methods may be implemented using any number of techniques, whether currently known or not yet in existence. The disclosure should in no way be limited to the illustrative implementations, drawings, and techniques illustrated below, but may be modified within the scope of the appended claims along with their full scope of equivalents.

The following brief definition of terms shall apply throughout the application:

The term “comprising” means including but not limited to, and should be interpreted in the manner it is typically used in the patent context;

The phrases “in one embodiment,” “according to one embodiment,” and the like generally mean that the particular feature, structure, or characteristic following the phrase may be included in at least one embodiment of the present invention, and may be included in more than one embodiment of the present invention (importantly, such phrases do not necessarily refer to the same embodiment);

If the specification describes something as “exemplary” or an “example,” it should be understood that refers to a non-exclusive example;

The terms “about” or approximately” or the like, when used with a number, may mean that specific number, or alternatively, a range in proximity to the specific number, as understood by persons of skill in the art field; and

If the specification states a component or feature “may,” “can,” “could,” “should,” “would,” “preferably,” “possibly,” “typically,” “optionally,” “for example,” “often,” or “might” (or other such language) be included or have a characteristic, that particular component or feature is not required to be included or to have the characteristic. Such component or feature may be optionally included in some embodiments, or it may be excluded.

Due to the extreme toxicity of H₂S gas, various countries have created regulations limiting the exposure of individuals to the gas. For example, the 2010 American Conference of Governmental Industrial Hygienists (ACGIH) has determined the H₂S safety value at an 8 hour time weighted average (TWA-8 hr.) of 1 ppm and a 15 min short term exposure (STEL-15 min) of 5 ppm. Various sensors have been developed to detect the presence and concentration of H₂S in the atmosphere. Electrochemical sensors using a noble metal catalyst to allow the H₂S to react to create a measurable current may also catalyze the reaction of other gas species such as CO to create an electrical current. For example, an ambient concentration of 10 ppm CO can create a cross-sensitivity of around 5 ppb of H₂S in a sensor having a low cross-sensitivity. However, the use of low cross-sensitivity sensors can experience slow response times, thereby making the sensors unfavorable in some situations.

Other sensors can have a faster response time, but may also have a higher cross-sensitivity to CO. For example, an ambient concentration of 50 ppm CO may create a cross-sensitivity reading of between about 0.5 to about 2.3 ppm H₂S. This reading may be sufficient to cause a false alarm for the 8 hour average and can create a false alarm for the short term exposure when higher concentrations of CO are present.

In order to address the cross-sensitivity of ambient gases in an electrochemical sensor for the detection of H₂S, a working electrode having a multi-layer design can be used in the H₂S sensor. The two layers can include a hydrophobic or hydrophobically treated substrate as the first layer and a second layer comprising a surface of the substrate treated with a surfactant. The surfactant can comprise a fluorosurfactant. This design substantially reduces the cross-sensitivity of the H₂S sensor to CO while exhibiting a relatively fast response time. The reduction in the cross-sensitivity can also reduce or eliminate false readings resulting from calibration offsets due to the low reactivity to CO.

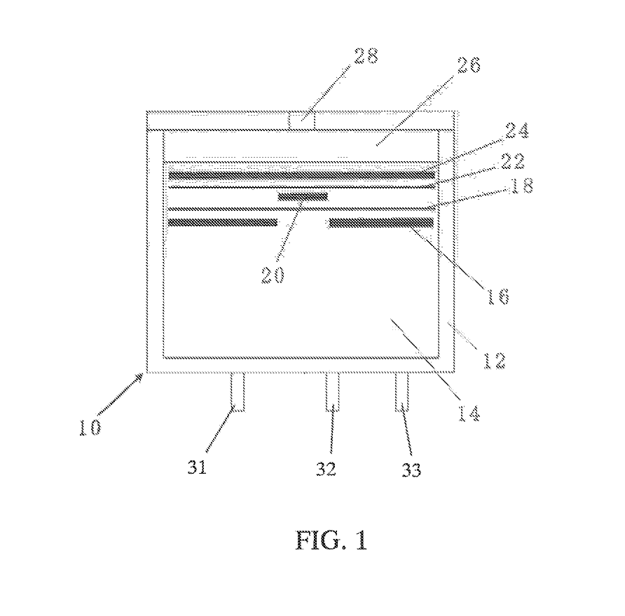

FIG. 1. is the cross section drawing of the electrochemical sensor 10. The sensor 10 generally comprises a housing 12 defining a cavity or reservoir 14 designed to hold an electrolyte solution. A working electrode 24 with a gas-permeable membrane can be placed between an opening 28 and the reservoir 14. The gas permeable membrane may allow the gas to be detected to enter the reservoir 14 and react with the working electrode 24. A counter electrode 16 and a reference electrode 20 can be positioned within the reservoir 14. When the gas reacts within the reservoir 14, an electrical current and/or potential can be developed between the electrodes to provide an indication of the concentration of the gas. A reference electrode 20 may also be positioned within the reservoir 14 to provide a reference for the detected current and potential between the working electrode 24 and the counter electrode 16.

The housing 12 defines the interior reservoir 14, and one or more openings 28 can be disposed in the housing to allow a gas to be detected to enter the housing 12 into a gas space 26. The housing 12 can generally be formed from any material that is substantially inert to the electrolyte and gas being measured. In an embodiment, the housing 12 can be formed from a polymeric material, a metal, or a ceramic.

The reservoir comprises the counter electrode 16, the reference electrode 20, and the working electrode 24. In some embodiment, the electrolyte can be contained within the reservoir 14, and the counter electrode 16, the reference electrode 20, and the working electrode 24 can be in electrical contact through the electrolyte. In some embodiments, a porous separator or other porous structure (e.g., a wick, etc.) can be used to retain the electrolyte in contact with the electrodes. The porous separator can be composed of polymer or glass fibers, for example, and can extend into the reservoir to provide the electrolyte a path to the electrodes. In an embodiment, a separator 18 can be disposed between the counter electrode 16 and the reference electrode 20, and a separator 22 can be disposed between the reference electrode 20 and the working electrode 24.

In an embodiment, the electrolyte may be in the form of a solid polymer electrolyte which comprises an ionic exchange membrane. In some embodiments, the electrolyte can be in the form of a free liquid, disposed in a matrix or slurry such as glass fibers (e.g., the separator 18, the separator 22, etc.), or disposed in the form of a semi-solid or solid gel.

The electrolyte can be any conventional aqueous acidic electrolyte such as sulfuric acid, phosphoric acid or a neutral ionic solution such as a salt solution (e.g., a lithium salt such as lithium chloride, etc.), or any combination thereof. For example, the electrolyte can comprise sulfuric acid having a molar concentration between about 6 M to about 10 M. Since sulfuric acid is hygroscopic, the concentration can vary from about 10 to about 70 wt % (1 to 11.5 molar) over a relative humidity (RH) range of the environment of about 3 to about 95%. As another example, the electrolyte can include a lithium chloride salt having about 30% to about 60% lithium chloride (LiCl) by weight, with the balance being an aqueous solution.

The working electrode 24 may be disposed within the housing 12. The gas entering the sensor 10 can contact one side of the working electrode 24 and pass through working electrode 24 to reach the interface between the working electrode 24 and the electrolyte. The gas can then react to generate the current indicative of the gas concentration. As disclosed herein, the working electrode 24 can comprise a plurality of layers. The base or substrate layer can comprise carbon and a hydrophobic material or a hydrophobically treated material. One side of the working electrode 24 can be treated with a surfactant and placed in contact with the electrolyte. This configuration may reduce the cross-sensitivity of the sensor 10 to the presence of carbon monoxide.

In an embodiment, the working electrode 24 can comprise a porous substrate as the base layer. The substrate can be electrically conductive and porous to the gas of interest, which can comprise hydrogen sulfide. The substrate can comprise a carbon paper formed of carbon or graphite fibers. The use of carbon may provide a sufficient degree of electrical conductivity to allow the current generated by the reaction of the gas with the electrolyte at the surface of the working electrode 24 to be detected by a lead coupled to the working electrode 24. Other electrically conductive substrates may also be used such as carbon felts, porous carbon boards, and/or electrically conductive polymers such as polyacetylene, each of which may be made hydrophobic as described below. In an embodiment, the substrate can be between about 5 mils to about 20 mils thick in some embodiments.

The porous substrate can be hydrophobic to prevent the electrolyte from passing through the working electrode 24. The substrate can be formed from a hydrophobic material, or the substrate can be treated with a hydrophobic material. In an embodiment, the substrate can be made hydrophobic through the impregnation of the substrate with a hydrophobic material such as a fluorinated polymer (e.g., polytetrafluoroethylene (PTFE), etc.). The impregnation process can include disposing a hydrophobic material containing solution or slurry on the substrate using a dipping, coating, or rolling process. Alternatively, a dry composition such as a powder can be applied to the substrate. In some embodiments, an optional sintering process can be used to infuse the hydrophobic material into the substrate to create the hydrophobic base layer for the working electrode 24, where both sides of the hydrophobic base layer are hydrophobic. The sintering process can cause the hydrophobic polymer to bond or fuse with the carbon of the substrate to securely bond the hydrophobic material to the substrate.

The resulting substrates can contain about 30% to about 50% by weight of the hydrophobic polymer. The amount of hydrophobic material added to the substrate can affect the electrical conductivity of the substrate, wherein the electrical conductivity tends to decrease with an increased amount of the hydrophobic material. The amount of the hydrophobic polymer used with the substrate may depend on the degree of hydrophobicity desired, the porosity to the hydrogen sulfide, and the resulting electrical conductivity of the working electrode.

When both sides of the substrate are hydrophobic, the working electrode 24 may not respond to the presence of hydrogen sulfide or carbon monoxide due to a limited interaction between a hydrophobic surface and the electrolyte. In order to allow the working electrode 24 to have a sensitivity to hydrogen sulfide, the surface of the substrate in contact with the electrolyte can be treated with a surfactant. The opposite side, which can be in contact with the gas passing through the opening 28, may be left hydrophobic.

The surfactant can be applied to one side of the substrate to form the working electrode 24 with two layers. The surfactant can be applied to the substrate as a solution by spraying, painting, coating, or the like. In an embodiment, the surfactant can include a fluorinated surfactant. Exemplary fluorinated surfactants include nonionic, amphoteric, and cationic fluorosurfactants. Suitable fluorosurfactants can include perfluoroalkylethyl methacrylate, perfluoroalkylethyl poly(ethyleneoxide)ethanol, 3-(perfluoroalkylethylthio) propionic acid lithium salt, a perfluoroalkyl sulfonate, and any combination thereof. In an embodiment, the surfactant may be Zonyl FSN-100, which comprises a perfluoroalkyl sulfonate and is available as a 40% solution in 50/50 water/isopropanol mixture from E.I. du Pont de Nemours & Co., Inc. of Wilmington, Del. The fluorosurfactant can be applied as a solution comprising between about 4% and about 10% by weight with a loading of between about 10 to about 20 μl per cm². The surfactant can be applied in a single coating or multiple coatings with the surfactant solution being allowed to dry between applications. Once the surfactant has been applied, the substrate can be dried to provide the working electrode 24 material. The material can then be cut or formed into the desired shape for the working electrode 24.

The two layer material used for the working electrode 24 can be disposed in the housing 12 so that the side having the surfactant disposed thereon is on contact with the electrolyte. The use of the working electrode 24 having the two-layer design may be useful in reducing the cross-sensitivity to carbon monoxide. It is believed that the reduced cross-sensitivity may be due to the carbon in the substrate reducing or eliminating the interference caused by the carbon monoxide while allowing the hydrogen sulfide to diffuse through the working electrode 24 to the electrolyte.

The counter electrode 16 can be disposed within the housing 12. The counter electrode 16 can comprise a hydrophobic membrane such as a PTFE membrane having a catalytic material disposed thereon. In an embodiment, the catalytic material, can be mixed with a hydrophobic material (e.g., PTFE, etc.) and disposed on the PTFE membrane. Any suitable process such as rolling, coating, screen printing, or the like can be used to apply the catalytic material on the membrane. The catalyst layer can then be membrane through a sintering process as described herein.

In an embodiment, the catalytic material can comprise a noble metal such as gold (Au), platinum (Pt), ruthenium (Ru), rhodium (Rh), Iridium (Ir), oxides thereof, or any combination thereof. In an embodiment, the catalytic material comprises a Pt—Ru mixture that is screen printed on the membrane.

Similarly, the reference electrode 20 can be disposed within the housing 12. The reference electrode 20 can comprise a hydrophobic membrane such as a PTFE membrane having a catalytic material disposed thereon. In an embodiment, the catalytic material, can be mixed with a hydrophobic material (e.g., PTFE, etc.) and disposed on the PTFE membrane. Any of the methods used to form the counter electrode can also be used to prepare the reference electrode 20. In an embodiment, the catalytic material used with the reference electrode 20 can comprise a noble metal such as gold (Au), platinum (Pt), ruthenium (Ru), rhodium (Rh), Iridium (Ir), oxides thereof, or any combination thereof. In an embodiment, the catalytic material used to form the reference electrode can comprise a Pt—Ru mixture that is screen printed on the membrane. While illustrated in FIG. 1 as having the reference electrode 20, some embodiments of the electrochemical sensor may not include a reference electrode 20.

In order to detect the current and/or potential difference across the electrodes in response to the presence of the hydrogen sulfide, one or more leads or electrical contacts can be electrically coupled to the working electrode 24, the reference electrode 20, and/or the counter electrode 16. The lead contacting the working electrode 24 can contact either side of the working electrode 24 since the substrate comprises an electrically conductive material. In order to avoid the corrosive effects of the electrolyte, the lead contacting the working electrode can contact the side of the working electrode 24 that is not in contact with the electrolyte. Leads may be similarly electrically coupled to the counter electrode 16 and the reference electrode 20. The leads can be electrically coupled to external connection pins 31, 32, 33 to provide an electrical connection to external processing circuitry. The external circuitry can detect the current and/or potential difference between the electrodes and convert the current into a corresponding hydrogen sulfide concentration.

The sensor 10 can be used to detect a hydrogen sulfide concentration in the presence of carbon monoxide. In use, the ambient gas can flow into the sensor 10 through the opening 28, which serves as the intake port for the sensor 10. The ambient gas can comprise hydrogen sulfide and/or carbon monoxide. The gas can contact the working electrode and pass through the fine pores of the porous substrate layer to reach the surface of the working electrode 24 treated with the surfactant. The electrolyte may be in contact with the surface of the working electrode 24 as a result of the surfactant treatment, and the hydrogen sulfide may react and result in an electrolytic current forming between the working electrode 24 and the counter electrode 16 that corresponds to the concentration of the hydrogen sulfide in the ambient gas. By measuring the current, the concentration of hydrogen sulfide can be determined using, for example, the external circuitry.

During the measurement process, an interfering gas such as the carbon monoxide can also contact the working electrode 24. The carbon monoxide can be adsorbed by the carbon in the substrate of the working electrode, which may prevent the carbon monoxide from reaching the electrolyte/working electrode 24 interface or at least reduce the amount of carbon monoxide reaching the interface. As a result, the sensor 10 may not produce a current in response to the presence of the carbon monoxide, or may have a reduced current output based on the presence of the carbon monoxide.

### EXAMPLES

The disclosure having been generally described, the following examples are given as particular embodiments of the disclosure and to demonstrate the practice and advantages thereof. It is understood that the examples are given by way of illustration and are not intended to limit the specification or the claims in any manner.

### Example 1

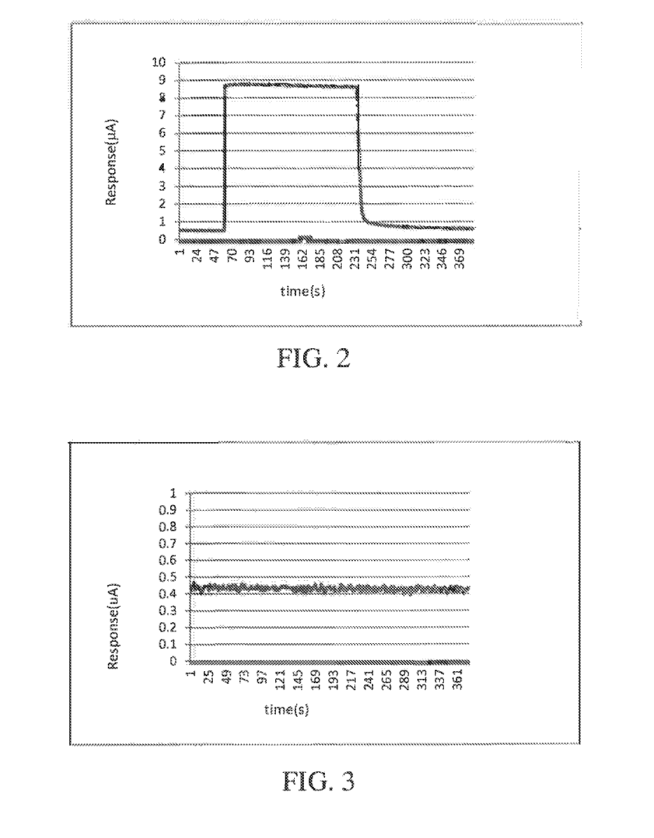

One side of a hydrophobic Tory carbon paper (whose two sides are hydrophobic) was treated with a 4% FSN-100 water solution. The FSN-100 solution could be applied using a brush or one side of the Tory carbon paper would be immersed in the solution. The side of the Tory carbon paper that was hydrophobic (i.e., not treated with the FSN solution) was placed on the gas side of the sensor. The treated side was placed in contact with the electrolyte. The H₂S sensor was assembled with 30% LiCl as the electrolyte. The reference and counter electrodes were prepared with PTFE and Pt:Ru as described herein, where the atomic ratio of Pt to Ru was about 1:2. The sensor was tested with a gas comprising 25 ppm H₂S. The resulting sensitivity was 0.33 μA/ppm. The T90 response time was 5 seconds and the baseline was 0.04 μA, where the resolution was about 0.1 ppm as shown in FIG. 2. The sensor was then exposed to 1 min air followed by 50 ppm CO. The CO cross sensitivity to H₂S was 0 ppm equivalent, as can be seen in FIG. 3.

### Example 2

The Tory carbon paper was prepared in the same way as described in Example 1. The H₂S sensor was assembled with 6M H₂SO₄ as the electrolyte. The reference and counter electrodes were prepared with PTFE and Pt:Ru as described herein, where the atomic ratio of Pt to Ru was about 1:2. The sensor was tested with 25 ppm H₂S. The resulting sensitivity was 0.06 μA/ppm. The T90 response time was 15 seconds and the baseline is 0.04 μA, where the resolution was about 0.1 ppm. The sensor was then exposed to air for 1 min, and then 50 ppm CO. The CO cross sensitivity to H₂S was 0 ppm equivalent.

While various embodiments in accordance with the principles disclosed herein have been shown and described above, modifications thereof may be made by one skilled in the art without departing from the spirit and the teachings of the disclosure. The embodiments described herein are representative only and are not intended to be limiting. Many variations, combinations, and modifications are possible and are within the scope of the disclosure. Alternative embodiments that result from combining, integrating, and/or omitting features of the embodiment(s) are also within the scope of the disclosure. Accordingly, the scope of protection is not limited by the description set out above, but is defined by the claims which follow, that scope including all equivalents of the subject matter of the claims. Each and every claim is incorporated as further disclosure into the specification and the claims are embodiment(s) of the present invention(s). Furthermore, any advantages and features described above may relate to specific embodiments, but shall not limit the application of such issued claims to processes and structures accomplishing any or all of the above advantages or having any or all of the above features.

Additionally, the section headings used herein are provided for consistency with the suggestions under 37 C.F.R. 1.77 or to otherwise provide organizational cues. These headings shall not limit or characterize the invention(s) set out in any claims that may issue from this disclosure. Specifically and by way of example, although the headings might refer to a “Field,” the claims should not be limited by the language chosen under this heading to describe the so-called field. Further, a description of a technology in the “Background” is not to be construed as an admission that certain technology is prior art to any invention(s) in this disclosure. Neither is the “Summary” to be considered as a limiting characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of the claims shall be considered on their own merits in light of this disclosure, but should not be constrained by the headings set forth herein.

Use of broader terms such as comprises, includes, and having should be understood to provide support for narrower terms such as consisting of, consisting essentially of, and comprised substantially of Use of the term “optionally,” “may,” “might,” “possibly,” and the like with respect to any element of an embodiment means that the element is not required, or alternatively, the element is required, both alternatives being within the scope of the embodiment(s). Also, references to examples are merely provided for illustrative purposes, and are not intended to be exclusive.

While several embodiments have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

1-15. (canceled)

16. An electrochemical H₂S sensor comprising:
a housing;
an electrolyte disposed within the housing; and
a plurality of electrodes in contact with the electrolyte within the housing, wherein the plurality of electrodes comprise a multi-layer porous working electrode, wherein the porous working electrode comprises a first surface layer that is hydrophobic and a second surface layer that is treated with a surfactant, wherein the first surface layer is exposed to an ambient gas, and wherein the second surface layer is in contact with the electrolyte.

17. The sensor of claim 16, wherein the multi-layer porous working electrode is electrically conductive.

18. The sensor of claim 16, wherein the multi-layer porous working electrode comprises a porous carbon paper.

19. The sensor of claim 18, wherein the porous carbon paper has a hydrophobic polymer impregnated therein.

20. The sensor of claim 16, wherein the surfactant comprises a fluorosurfactant.

21. The sensor of claim 16, wherein the surfactant comprises a perfluoroalkylehtyl methacrylate, a perfluoroalkylethyl poly(ethyleneoxide)ethanol, a 3-(Perfluoroalkylethylthio) propionic acid lithium salt, a perfluoroalkyl sulfonate, or any combination thereof.

22. The sensor of claim 16, wherein the surfactant is a perfluoroalkyl sulfonate.

23. The sensor of claim 16, wherein the electrolyte comprises LiCl having a concentration of between about 30% to about 60% by weight.

24. The sensor of claim 16, wherein the electrolyte comprises sulfuric acid having a concentration between about 6 M to about 10 M.

25. The sensor of claim 16, wherein the plurality of electrodes comprises a counter electrode, wherein the counter electrode comprises a mixture of PTFE and Pt—Ru disposed on a PTFE membrane.

26. The sensor of claim 16, wherein the plurality of electrodes comprises a reference electrode, wherein the reference electrode comprises a mixture of PTFE and Pt—Ru disposed on a PTFE membrane.

27. An electrochemical H₂S sensor comprising:
a housing;
an electrolyte disposed within the housing;
a reference electrode disposed within the housing and in contact with the electrolyte;
a counter electrode disposed within the housing and in contact with the electrolyte; and
a multi-layer porous working electrode, wherein the multi-layer porous working electrode comprises a substrate comprising carbon and a hydrophobic material, wherein a first surface layer of the substrate is hydrophobic, wherein a second surface layer of the substrate opposite the first surface layer is treated with a fluorosurfactant, wherein the first surface layer is exposed to an ambient gas, and wherein the second surface layer is in contact with the electrolyte.

28. The sensor of claim 27, wherein the counter electrode comprises a mixture of PTFE and Pt—Ru disposed on a PTFE membrane.

29. The sensor of claim 27, wherein the reference electrode comprises a mixture of PTFE and Pt—Ru disposed on a PTFE membrane.

30. The sensor of claim 27, wherein the surfactant comprises a perfluoroalkylehtyl methacrylate, a perfluoroalkylethyl poly(ethyleneoxide)ethanol, a 3-(Perfluoroalkylethylthio) propionic acid lithium salt, a perfluoroalkyl sulfonate, or any combination thereof.

31. A method of detecting hydrogen sulfide, the method comprising:
receiving an ambient gas into a housing, wherein the ambient gas comprises hydrogen sulfide;
contacting the ambient gas with a first surface layer of a multi-layer porous working electrode, wherein the multi-layer porous working electrode comprises a substrate comprising carbon and a hydrophobic material, wherein the first surface layer of the substrate is hydrophobic, wherein a second surface layer of the substrate opposite the first surface layer is treated with a fluorosurfactant;
allowing the hydrogen sulfide to diffuse through the multi-layer porous working electrode to contact an electrolyte;
generating a current between the multi-layer porous working electrode and a counter electrode in response to a reaction between the hydrogen sulfide and the electrolyte at the second surface layer of the multi-layer porous working electrode; and
determining a concentration of the hydrogen sulfide in the ambient gas based on the current.

32. The method of claim 31, wherein the ambient gas further comprises carbon monoxide, and wherein the method further comprises:
adsorbing the carbon monoxide on the carbon in the substrate; and
preventing at least a portion of the carbon monoxide from contacting the electrolyte through the multi-layer porous working electrode.

33. The method of claim 31, further comprising placing the multi-layer porous working electrode between an opening of the housing and a reservoir of the housing containing the electrolyte.

34. The method of claim 31, further comprising placing the first surface layer of the multi-layer porous working electrode in contact with the ambient gas, and placing the second surface layer of the multi-layer porous working electrode in contact with the electrolyte.

35. The method of claim 16, wherein the multi-layer porous working electrode comprises a porous carbon paper, and wherein the hydrophobic material comprises a hydrophobic polymer impregnated in the porous carbon paper.
